(19) United States
(12) Patent Application Publication
(10) Pub. No.: US 2026/0026958 A1
(43) Pub. Date: Jan. 29, 2026

(54) OSTOMY BAG SYSTEM

(71) Applicant: Ostique Limited, London (GB)

(72) Inventors: Stephanie MONTY, London (GB); Toni SCHNEIDER, London (GB); Austen Charles MILLER, Whitchurch, Hampshire (GB); Daniel PHILLIPS, Whitchurch, Hampshire (GB); Thomas Henry SUSSEX, Whitchurch, Hampshire (GB)

(73) Assignee: Ostique Limited, London (GB)

(21) Appl. No.: 18/998,643

(22) PCT Filed: Jul. 26, 2023
(86) PCT No.: PCT/GB2023/051976

§ 371 Date: Jan. 27, 2025

(30) Foreign Application Priority Data
Jul. 26, 2022 (GB) 2210932.6

(51) Int. Cl. A61F 5/443; A61F 5/441

(52) U.S. Cl. CPC A61F 5/443; A61F 5/441

(57) ABSTRACT

There is provided an adhesive wafer for an ostomy bag system, comprising a layer, the layer comprising a plurality of formations at an outer part of the layer, wherein each formation comprises an overhanging portion.

### INTRODUCTION

The present invention relates to an ostomy bag system. In particular, it relates to an adhesive wafer and a filter system for implementation in an ostomy bag system.

### BACKGROUND

Ostomy is a common surgical procedure during which an artificial opening, known as a stoma, is created for the discharge of bodily wastes. Patients who have undergone such a procedure, known as ostomates, typically wear products such as ostomy bags to collect the bodily wastes. The ostomate must empty and change the ostomy bag regularly, so it is important that this process is as comfortable and efficient as possible.

The present invention seeks to provide improved components to enhance the comfort with which a user may wear and clean an ostomy bag system.

### STATEMENTS OF INVENTION

Aspects of the disclosure are set out in the accompanying claims.

According to a first aspect of the invention, there is provided an adhesive wafer for an ostomy bag system, comprising a layer, the layer comprising a plurality of formations at an outer part of the layer, wherein each formation comprises an overhanging portion.

In this context, ‘overhanging portion’ is used to mean a portion which extends/protrudes beyond the formation, where there is a gap between the extending portion and a main body (the layer). In the present instance, the layer (including the plurality of formations) is formed as a generally planar/flat element. The extension of the ‘overhanging portion’ preferably remains generally within the plane (or in other words, the term ‘overhanging’ in this context preferably does not refer to an element which is above the plane of the layer). The overhanging portion preferably extends in a direction which is not directly outwards (relative to a centre of the layer). The formations (and the overhanging portions) are preferably made of the same materials as the layer. The layer and the formations (and the overhanging portions) are preferably a unitary component.

The described arrangement can provide improved conformability to the user's body, as the ‘swept’ nature of the formations including overhanging portions can allow the formations to move independently or at least semi-independently of one another to conform over the surface of the body. The described arrangement can also improve adherence, as it can prevent the wafer from becoming unstuck when a user moves.

The layer may comprise a plurality of cut-outs extending into the layer, wherein at least a portion of each of the plurality of cut-outs extends into the layer in a direction which is not directly towards a centre of the layer.

As used herein, the term ‘cut-out’ preferably connotes a portion cut away from a layer. As used herein, the term ‘centre’ preferably connotes a geometrical central region or point. This arrangement can provide enhanced conformability to uneven body surfaces, and improved adherence and security. This is because the portions between the cut-outs can move at least partially independently of one another. In other words, the cut-outs provide ‘relief’ of stresses that would otherwise exist at the join between portions. The extension of the cut-outs in a direction which is not directly towards a centre of the layer can increase their ‘effective length’, thereby improving separation of the portions between the cut-outs, while preventing the need for the cut-outs to extend too far towards the centre (which can lead to weak areas of adhesion). The arrangement thereby reduces the risk of leaks occurring.

The direction in which the cut-outs extend may optionally be defined, using a polar coordinate system centred on the centre of the layer, as being not directly in a (inward) radial direction. The direction may be defined as a vector with a non-zero tangential component. The above mention of a polar coordinate system should not be considered as limiting the layer to having only a circular or generally circular shape-it will be appreciated that other shapes are possible.

(At least part of) each overhanging portion may be separated from the layer by (at least part of) a respective cut-out. Aligning the overhanging portions and cut-outs may allow the overhanging portions and/or the formations to move more independently from the main body of the layer. The shape of the cut-outs may define the shape of the formations and/or the shape of the overhanging portions, or vice versa. The ‘formations’ may also be referred to as ‘petals’, but they are not limited to being the shape of flower petals (or similar).

According to another aspect of the invention, there is provided an adhesive wafer for an ostomy bag system, comprising a layer, the layer comprising a plurality of cut-outs extending into the layer, wherein at least a portion of each of the plurality of cut-outs extends into the layer in a direction which is not directly towards a centre of the layer. The layer may comprise a plurality of formations at an outer part of the layer, wherein each formation may comprise an overhanging portion.

(At least part of) an outer edge of each of the formations may be shaped as parts of a spiral (i.e. parts of a tightening curve centred on an axis, where said axis may be coincident with a centre of the layer). That is, the outer edge of the formations may have a variable radius relative to a centre of the layer, preferably such that the outer edge of the formations has a curved shape. The overhanging portion in respect of a particular formation may be formed at or towards an end of a formation having at least one of the following characteristics: the end is relatively further away from a centre of a layer (than another end); and the outer edge of the end has a larger radius (than another end).

The formations may be arranged evenly around the layer, preferably wherein the formations are arranged so as to be rotationally symmetrical. Preferably each formation is non-circular. Each formation is preferably the same size. There are preferably provided between 4 and 7 formations, and more preferably 5 or 6, where this number may avoid each formation have an excessive arc length which may lead to problems in adhesion. Preferably, each formation has a curved outer edge encompassing the overhanging portion. The curved outer edge may have a radius of curvature smaller than the radius of curvature of the circular arrangement. This can help to prevent lifting of the formations when worn by a user. It can also allow greater flexibility and independence of movement. In some instances, the radius of curvature of the curved outer edge is at an acute angle to the radial direction of the layer or the first layer of between 5° and 10° degrees, preferably between 6° and 9°, more preferably between 6.5° and 8.5°, even more preferably between 7° and 8°. This can create a ‘swept’ arrangement of the formations. This can improve the extent to which the formations can move at least semi-independently of one another. The curved outer edge may have a radius of curvature smaller than the width of the layer, preferably wherein the curved outer edge has a radius of curvature smaller than the radius of the layer. This creates a smaller curve than the overall outline of the layer. This can further improve the extent to which the formations can move at least semi-independently of one another.

Preferably, the overhanging portions do not extend over adjacent formations. This assists in allowing the layer to conform over uneven surfaces. It can also aid in allowing the formations to move independently.

The overhanging portions may have a rounded end, preferably wherein the overhanging portions have a partially circular end, preferably wherein the partially circular end has a radius of curvature between 2 mm and 4 mm, more preferably of approximately 3 mm. The rounded end can help to prevent the overhanging portion from becoming unstuck and peeling off. This is because there is a smooth transition surface at the edge. Wrinkles can often occur at sharp edges, and this arrangement can assist in preventing wrinkles from occurring.

The layer may comprise a plurality of cut-outs having curved ends. This can help to prevent tears occurring at the ends of the cut-outs. It can also assist in maintaining adhesion by avoiding any sharp angles which could provide initiation points for wrinkles. Preferably, the curved ends have a generally semi-circular shape. The curved ends may have a radius of curvature of between 2 and 3 mm, preferably between 2 and 2.5 mm. A radius of curvature of a partially circular end of each overhanging portion may be greater than a radius of curvature of a curved end of a cut-out.

The outermost points of the layer may be arranged in a circle. That is, the layer may have a generally circular shape/an approximately circular outline/a generally circular footprint. This means the footprint of the layer is approximately circular if the cut-out portions and/or the formations are discounted. The formations are preferably arranged in a circular arrangement around the layer. The formations may be arranged in different orientations facing outwards around the circular arrangement. This can allow the formations to conform to the user's body. Preferably, each formation extends over a circular sector of the layer or the first layer having a central angle of approximately 72 degrees. Each formation preferably extends over an equal circular sector of the layer. Each overhanging portion may extend over a circular sector of the layer or the first layer having a central angle of between 2 and 6 degrees, preferably between 3 and 5 degrees, more preferably approximately 4 degrees. The shape of the layer may have rotational symmetry. The shape of the layer may have rotational symmetry but not reflective symmetry.

The cut-outs extend into the layer in a direction which is at an acute angle to a radius of the layer, preferably wherein said angle is between 15 and 45 degrees, more preferably wherein said angle is between 25 and 35 degrees, yet more preferably wherein said angle is approximately 30 degrees. Use of such an angle may help to increase the effective length of the cut-out, which can improve the extent to which the formations can move independently (or at least partially independently) of one another.

The cut-outs may extend into the layer by not more than 30% of the width (i.e. the circumference, specifically the maximum circumference including the formations) of the layer, preferably between 15 to 25%, more preferably by approximately 20%. This can reduce the likelihood of a leak or just a wrinkle occurring between the centre of the layer and the cut-out. The extension of the cut-outs in the radial direction may be less than 20% of the width (i.e. the circumference, specifically the maximum circumference including the formations) of the layer, preferably approximately 10%.

The width of the overhanging portions (at their widest point) may be 10% to 20% of the radius of the layer (in the radial direction), more preferably 12% to 18%, even more preferably 14% to 16%, preferably approximately 15% of the radius of the layer. The radius of the layer may be approximately 4 cm to 8 cm, preferably 4 cm to 6 cm, more preferably approximately 5 cm. The width of the overhanging portion in the radial direction may be 0.6 cm to 1.2 cm, preferably 0.6 cm to 1 cm, more preferably approximately 0.8 cm. The cut-outs may extend into the layer or the first layer such that a distance between the aperture and each cut-out is no less than 2 cm, preferably no less than 2.5 cm, more preferably no less than 3 cm. This can help to prevent ‘tunnelling’ between the aperture and the end of the cut-out, and can help to prevent a leak from occurring between the aperture and the end of the cut-out. The cut-outs may have a width between 2 mm and 7 mm, preferably between 3 mm and 6 mm, and more preferably of approximately 5 mm. This can allow sufficient separation between formations and/or the overhanging portions and the layer or the first layer, without being so wide as to lead to areas of weakness in adhesion. The number of cut-outs may be between 4 and 7 cut-outs, preferably between 4 and 6, and more preferably 5. If there are too few cut-outs, the flexibility and conformability of the wafer may be reduced, as the ability of the formations to move at least semi-independently may be reduced. If there are too many cut-outs, the adhesion of the wafer may be compromised.

The adhesive wafer may further comprise a central aperture located at a central position on the layer. This can be placed over a stoma, such that the adhesive wafer can be fitted around it.

The adhesive wafer may include a further (second) layer, wherein: the (first) layer has a greater thickness than the further (second) layer, and/or the further (second) layer has a dimension greater than that of the (first) layer, such that it extends beyond the outer part of the (first) layer.

According to a further aspect of the invention, there is provided an adhesive wafer for an ostomy bag system, formed as a wafer comprising at least a first layer and a second layer, wherein a first layer comprises a plurality of formations at an outer part of the layer and the second layer has a planar dimension greater than that of the first layer, such that it extends beyond the outer part of the first layer. This arrangement may allow the first layer to be configured to have skin compatible properties, while the second layer can be configured to provide good adhesive properties. The skin closest to a stoma is likely to be the most sensitive, and so it is advantageous to enhance the skin compatible properties in this area. The plurality of formations on the first layer can aid the conformability of the adhesive wafer. The first layer may have a greater thickness than the second layer.

According to a further aspect of the invention, there is provided an adhesive wafer for an ostomy bag system, formed as a wafer comprising at least a first layer and a second layer, wherein the first layer has a greater thickness than the second layer. This can allow for the inclusion of materials designed to regulate excess moisture and maintain healthy skin. The thinner second layer can improve flex and therefore conformability. The second layer may have a thickness of 20% to 40% of the first layer, preferably of 25% to 35% of the first layer. The thinner second layer can assist in improving the conformability of the wafer. This arrangement also leads to an effectively tapered edge, which can improve the longevity of the adhesion of the wafer.

According to a further aspect of the invention, there is provided an adhesive wafer for an ostomy bag system, formed as a wafer comprising at least a first layer and a second layer, wherein the first layer and second layer have different adhesive properties. Preferably, the first layer and second layer comprise different adhesives and/or the first layer and second layer have different adhesive strengths.

The (first) layer may be configured to be adhered to skin. The further (second) layer may be configured to be adhered to skin. The (first) layer may comprise adhesive. The further (second) layer may comprise adhesive. The (first) layer and further (second) layer may comprise different adhesives. Preferably, the second (further) layer may be more adhesive than the (first) layer (i.e. may have higher adhesive strength). The further (second) layer may comprise an adhesive, where preferably the layer or first layer comprises a different adhesive (a ‘further adhesive’). The adhesive and further adhesive may have different adhesive strengths and/or moisture-regulating properties.

The first layer may be formed of and/or comprise a hydrocolloid. This may improve regulation of excess moisture and assist in maintaining healthy skin. The first and second layer are preferably arranged adjacent to each other and/or in contact with each other. Preferably the layer or the first layer has a diameter of between 8 cm and 15 cm, more preferably between 9 cm and 12 cm, and even more preferably approximately 10 cm.

In preferable implementations, the first layer comprises a plurality of formations at an outer part of the layer, wherein each formation comprises an overhanging portion. The first layer may comprise a plurality of cut-outs extending into the layer, wherein at least a portion of each of the plurality of cut-outs extends into the layer in a direction which is not directly towards a centre of the layer. This can improve conformability of the wafer and the longevity of adhesion.

The wafer may be arranged such that the or a second layer is configured to be attached to an ostomy bag. The ostomy bag can collect waste matter. The adhesive wafer may be permanently attached, for example by bonding or welding to the ostomy bag. Alternatively, there may be provided a reversible coupling mechanism between the adhesive wafer and the ostomy bag. The adhesive wafer is preferably arranged to be adhered to a user's skin in use (thereby to adhere an ostomy bag system to the user). In preferable implementations, the layer/first layer is formed of a hydrocolloid. This is generally a skin-compatible choice with good adherence.

According to a further aspect of the invention, there is provided an adhesive wafer comprising a plurality of portions separated by cut-outs, wherein at least a portion of the plurality of portions are shaped such that they extend a greater distance from a centre of the wafer adjacent a cut-out. This arrangement can increase the effective length of the cut-outs for a particular size of wafer. This can improve the extent to which the portions between the cut-outs can move at least semi-independently, without the need for the cut-outs to extend further into the wafer. In this arrangement, the cut-outs may have a direction directly towards the centre of the layer, or they may have a direction having a tangential component. The portions are each adjacent to two cut-outs. Each portion may extend a greater distance from a centre of the wafer adjacent to one or both of these cut-outs. This arrangement may further comprise any of the features as outlined above.

According to a further aspect of the invention, there is provided an adhesive wafer for an ostomy bag system, comprising a layer, the layer comprising a plurality of formations at an edge of a layer, wherein at least a portion of each formation extends in a direction that is not directly away from the centre of the layer.

According to a further aspect of the invention, there is provided an adhesive wafer for an ostomy bag system comprising a plurality of formations at an outer part of the layer, wherein at least part of an outer edge of each formation is shaped as a partial spiral/have a curved shape with a gradually increasing radius, preferably where the formations are rotationally symmetrical.

According to a further aspect of the invention, there is provided an adhesive wafer for an ostomy bag system comprising a layer, the layer comprising a plurality of formations at an outer part of the layer, wherein the layer has a circular shape (less the formations) and each of the plurality of formations have an outer edge with a varying radius.

According to a further aspect of the invention, there is provided a filter system for an ostomy bag comprising a first aperture provided on a first (internal) surface, a second aperture provided on an second (external) surface and a filter, wherein the filter is provided between the first surface and the second surface and between the first aperture and the second aperture, such that the first aperture and the second aperture are located at different ends of a length (i.e. a longest dimension) of the filter.

This arrangement can facilitate in-plane filtering. This can increase the distance over which a gas travels through filtering material without the need for an increased thickness of filtering material. This can improve the filtering capability without adding bulk. The filter may have a curved shape (preferably along its length), for space-efficiency.

Preferably, the first aperture and/or second aperture comprise a semi-permeable membrane. This can prevent the ingress of unwanted fluids, which could clog the filter. Preferably, the first aperture and/or second aperture comprise(s) a semi-permeable membrane having a low-friction coating. This can prevent waste material from becoming attached to the surface, so that gas can continue to pass through the first semi-permeable membrane towards the filter. This can prevent water or other material from becoming attached to the second semi-permeable membrane. The filter may comprise activated carbon. This can improve the deodorising capability of the filter.

Preferably, the filter system comprises a cover to contain and/or fluidly seal the filter (other than at the apertures). As used herein, this preferably connotes that the filter is fluidly sealed other than via the apertures. This can ensure that the only possible pathway for a gas is to travel through the filter. It can also prevent the filter from becoming clogged.

Preferably, the filter is bonded to the internal and/or external surface by a glue arranged in a lattice formation. This can ensure secure attachment, while still allowing gases to pass through.

The filter system may be located proximate to an upper edge of the ostomy bag when in use. This can allow gases to pass through. This can also prevent liquid or solid waste matter from clogging the filter, or blocking the ingress of gas to the filter system.

According to a further aspect of the invention, there is provided an ostomy bag, comprising a means or formation arranged to be held by a user so as to temporarily occlude an opening in the ostomy bag. The opening is preferably an opening in or adjacent to a resealable draining spout at the bottom of the ostomy bag (when in use).

This can allow a user to apply pressure across a longer dimension by pressing down on only one part of the means or formation. Such a means or formation may be referred to as a ‘pressure strip’. A ‘pressure strip’ is configured to transmit pressure from a point, and apply it to a larger area.

According to a further aspect of the invention, there is provided an ostomy bag, comprising a means or formation configured to transmit pressure across its surface area upon application of pressure to the means or formation at any position on its surface.

According to a further aspect of the invention, there is provided an ostomy bag, comprising a pressure strip configured to temporarily occlude an opening. This can allow a user to prevent or limit the passing of matter through the opening, which can be advantageous for, for example, cleaning.

Preferably, the means or formation (or ‘pressure strip’) is formed of rigid or semi-rigid strip of material, such as plastic. This can help to achieve the desired pressure transmission. The means or formation may be mounted on an outer part of the ostomy bag and/or extend coincident to the width of the opening, preferably such that when held by the user the means or formation applies pressure over the width of the opening.

The ostomy bag may further comprise a draining spout extending from the opening. This can facilitate the draining of waste matter from the bag (through the opening and through the draining spout). The draining spout can direct the waste matter as it is drained out, so that it can be easily disposed of. The draining spout may be configured to be folded into a sealed configuration; preferably wherein the spout comprises wings which form part of a fastening mechanism. The spout may be configured as an elongate portion. This can allow a user to fold the spout up, and the bag can securely collect waste matter. When the user wishes to drain the bag, they can unroll the spout again.

The distal end of the spout may be formed of a rigid or semi-rigid material. The distal end may have a higher rigidity than the ostomy bag and/or a main portion of the spout. This can improve the ease with which the spout can be rolled, and help to improve the stability of the rolled-up configuration.

A distal end of the spout may be configured to be moveable between a flat configuration and an open configuration, preferably wherein the distal end of the spout is formed of a rigid or semi-rigid material; more preferably wherein the distal end comprises fold lines. This can aid in keeping the spout open when a user wishes to drain the bag, and to keep it closed and secure when the user wishes to roll up the spout.

The fastening mechanism may comprise a hook and loop fastening mechanism. This can facilitate easy reversible securement. In preferable implementations, the ostomy bag further comprises a cover configured to be placed over the spout, preferably configured to cover the spout in the sealed configuration. This can smooth the surface of the spout, which can help to improve the comfort of the wearer. It can also help to keep the bag less conspicuous under a user's clothes.

Preferably, the spout is formed of a wipe-clean material. This can improve the ease with which the spout can be cleaned. The ostomy bag may be formed of a wipe-clean material.

The ostomy bag preferably further comprises an aperture configured to be fluidly connected to a stoma in use, wherein the opening is configured to be located below the aperture when in use. Waste matter from the stoma can be collected in the bag. This can facilitate the waste matter travelling downwards towards the opening, through which the bag can be drained.

In preferable implementations, the bag comprises a tapered pouch, preferably wherein the tapered pouch is located adjacent to the opening. This can encourage waste matter to collect in this region of the bag. The waste matter can then be drained through the opening (for example, through the spout).

According to a further aspect of the invention, there is provided an ostomy bag system comprising at least one of: the wafer as described above; the filter system as described above; and the ostomy bag as described above. These can be provided in any combination.

In some implementations, the ostomy bag may be attachable to the baseplate via a reversible coupling mechanism.

As used herein, the term ‘wafer’ preferably connotes a thin component. This may encompass one or more layers. The wafer may be generally flat or have a concave or convex shape. The term ‘wafer’ may be used interchangeably with the word ‘patch’ and other similar such terms.

The invention extends to methods and/or apparatus substantially as herein described with reference to the accompanying drawings.

The term ‘comprising’ as used in this specification and claims preferably means ‘consisting at least in part of’. When interpreting statements in this specification and claims which include the term ‘comprising’, other features besides the features prefaced by this term in each statement can also be present. Related terms such as ‘comprise’ and ‘comprised’ are to be interpreted in a similar manner.

It will be appreciated that any dimensions described herein could be suitably scaled up or down for use in a larger or smaller version fo eth described device.

Any apparatus feature as described herein may also be provided as a method feature, and vice versa.

Any feature in one aspect of the invention may be applied to other aspects of the invention, in any appropriate combination. In particular, method aspects may be applied to apparatus aspects, and vice versa. Furthermore, any, some and/or all features in one aspect can be applied to any, some and/or all features in any other aspect, in any appropriate combination.

It should also be appreciated that particular combinations of the various features described and defined in any aspects of the invention can be implemented and/or supplied and/or used independently.

These and other aspects of the present invention will become apparent from the following exemplary embodiments that are described with reference to the following figures in which:

### BRIEF DESCRIPTION OF FIGURES

FIG. 1a shows a front perspective view of an ostomy bag system according to an embodiment of the present invention, in which the spout is in an unrolled configuration;

FIG. 1b shows a back perspective view of the ostomy bag system of FIG. 1a, in which the spout is in the unrolled configuration;

FIG. 2a shows a front perspective view of the ostomy bag system of FIGS. 1a and 1b, in which the spout is in a rolled-up configuration;

FIG. 2b shows a back perspective view of the ostomy bag system of FIGS. 1a, 1b and 2a, in which the spout is in the rolled-up configuration;

FIG. 3a shows a back view of an ostomy bag system according to an embodiment of the present invention, in which the spout is in the unrolled configuration;

FIG. 3b shows a side view of the ostomy bag system of FIG. 3a;

FIG. 4 shows an exploded perspective view of an ostomy bag and baseplate;

FIG. 5a shows a side view of a baseplate;

FIG. 5b shows a back view of a baseplate;

FIG. 5c shows an exploded perspective view of a baseplate;

FIG. 5d shows a front perspective view of a baseplate;

FIG. 5e shows a further back view of a baseplate including exemplary dimensions;

FIG. 6a shows a back view of an ostomy bag according to an embodiment of the present invention;

FIG. 6b shows a front view of the ostomy bag of FIG. 6a;

FIG. 7 shows a perspective exploded view of a front sheet of an ostomy bag and the components of a filter system;

FIG. 8 shows a schematic cross-section of a filter system according to the present invention;

FIG. 9 shows a section of a filter component and a glue lattice;

FIG. 10 shows a perspective exploded view of a front sheet of an ostomy bag and the components of a resealable spout;

FIG. 11a shows a tailpiece support in a linear configuration;

FIG. 11b shows the tailpiece support of FIG. 11a in a bent configuration;

FIG. 12a shows a back view of an ostomy bag system comprising a retaining flap;

FIG. 12b shows a perspective exploded view of a back sheet of an ostomy bag, a retaining ring and a retaining flap;

FIG. 12c shows a front view of a retaining flap;

FIG. 13 shows an alternative embodiment of a central layer of a baseplate; and

FIG. 14 shows an alternative arrangement of the layers of a baseplate.

### SPECIFIC DESCRIPTION

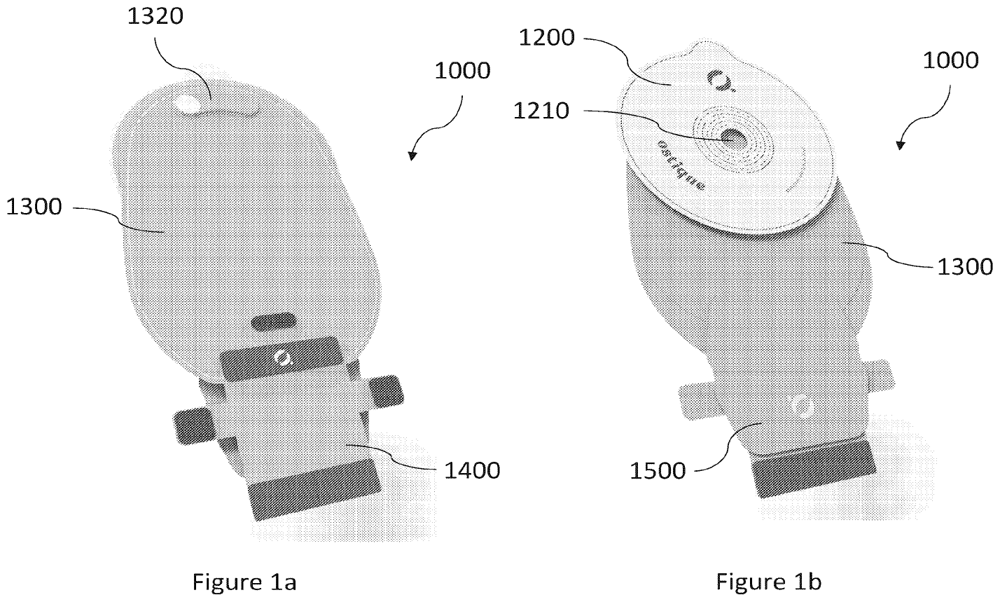

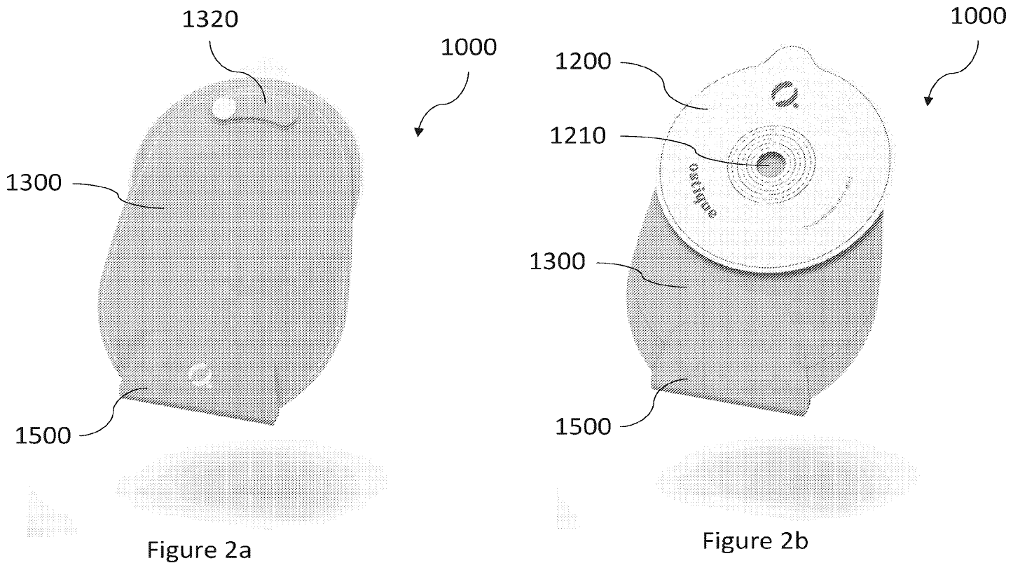

FIGS. 1a and 1b show perspective views of the front and back, respectively, of an example of an ostomy bag system 1000 of the present invention in an ‘unrolled’ or extended configuration. FIGS. 2a and 2b show perspective views of the front and back, respectively, of the example of an ostomy bag system 1000 of the present invention in a rolled-up or ‘compact’ configuration. The ostomy bag system 1000 comprises a baseplate 1200, and a bag 1300 connected to a spout 1400, which can be covered by a cover or ‘retaining flap’ 1500. The baseplate 1200 is formed as a generally planar circular disc-shaped plate which is configured to be attached to the body of a user, and contains an aperture 1210 configured to be placed over a stoma (such that the stoma extends through the aperture 1210). The aperture 1210 is fluidly connected to the interior of the bag 1300 via a further aperture 1330 in the bag 1300, such that bodily waste discharged from the stoma can pass through the baseplate aperture 1210 into the bag 1300. The assembly of the spout 1300 enables bodily waste to be securely collected when the system 1000 is in the ‘rolled-up’ or ‘compact’ configuration (as shown in FIGS. 2a and 2b). The spout 1400 can be unrolled to enable bodily waste to be drained out of the spout 1400.

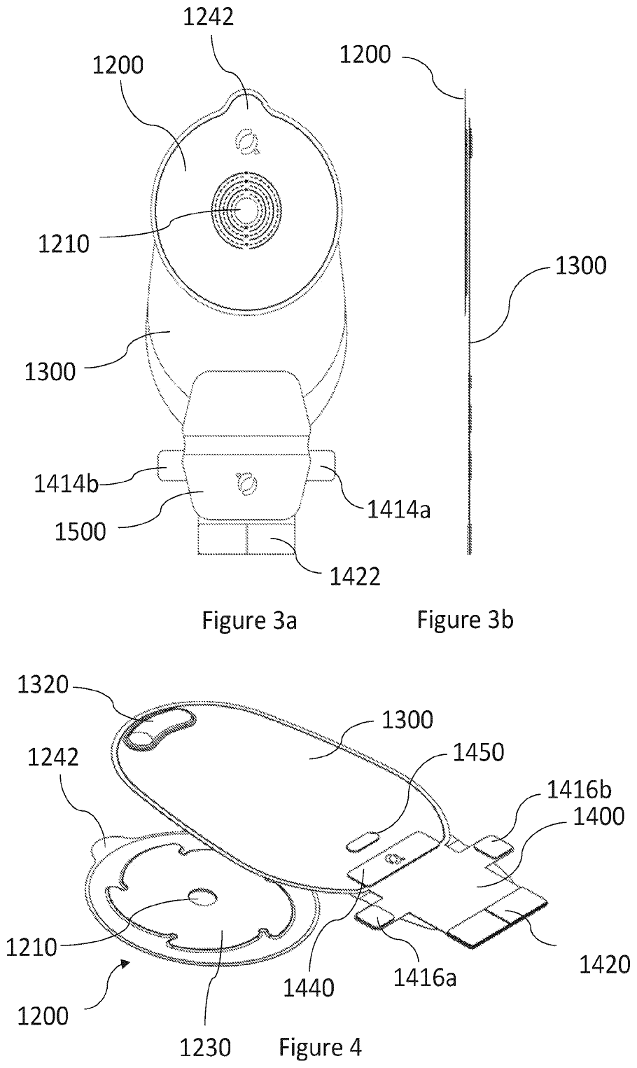

FIG. 3a shows a back view of the ostomy bag system 1000, i.e. the side which is configured to be placed against a user's body. FIG. 3b shows a side view of the ostomy bag system 1000. The ostomy bag system 1000 is configured to sit against the body of a wearer under their clothes, and so (when first used) the bag system 1000 has a generally planar flat shape, so that the ostomy bag device may be inconspicuous under a user's clothes.

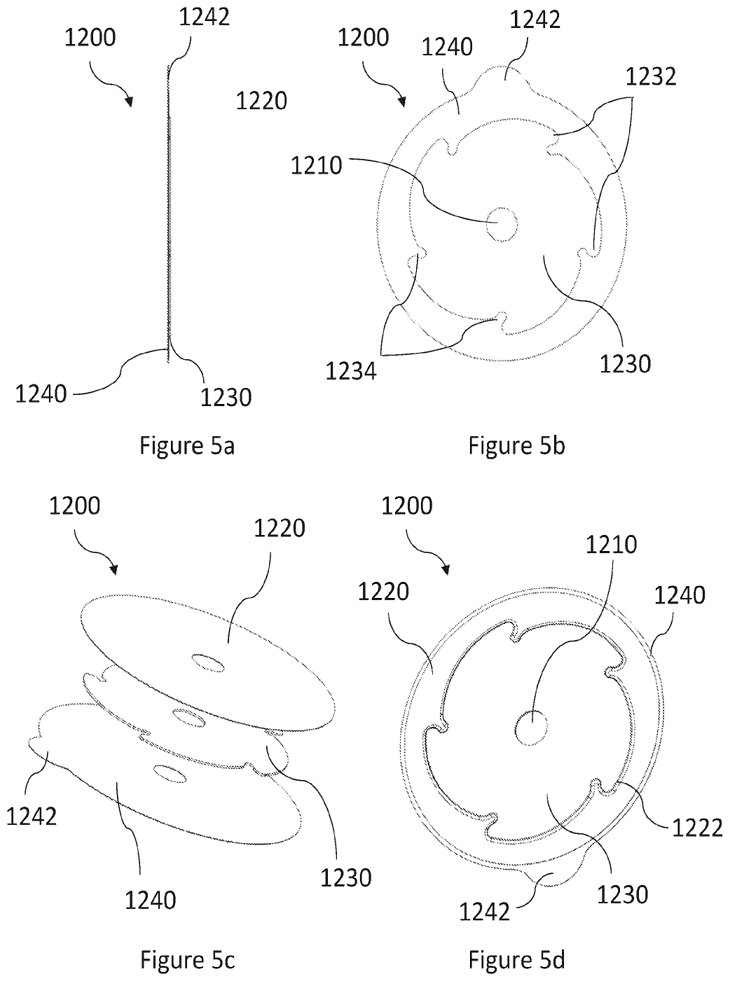

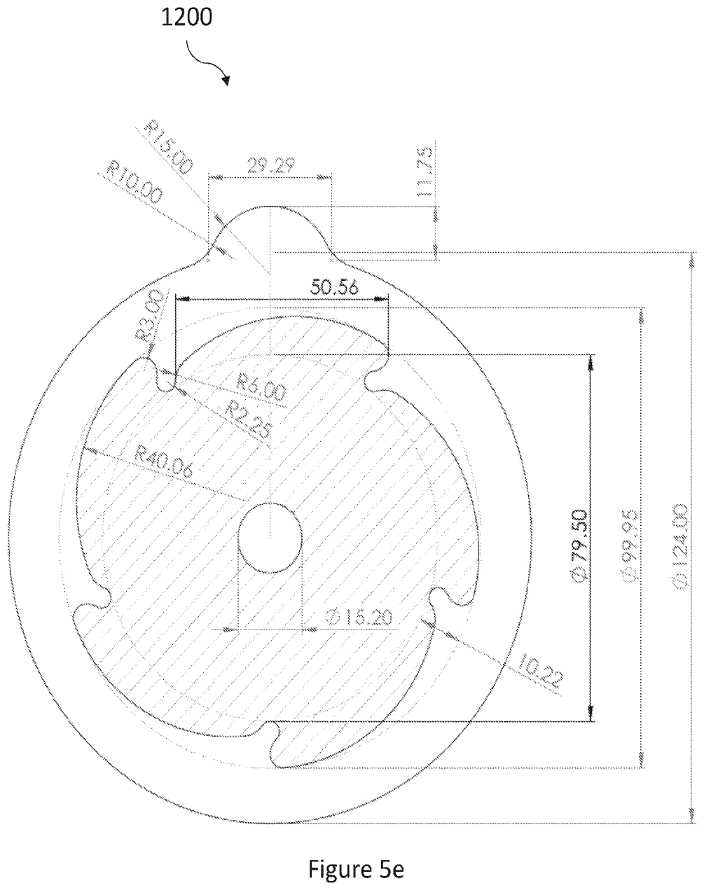

The baseplate 1200 is attached to the bag 1300 to form a one-piece ostomy bag system 1000. This may be a permanent attachment, for example via bonding, or via a reversible coupling mechanism such as that described in applicant's previous application, WO2021/220019, the disclosure of which is herein incorporated by reference. The expected behaviour is that users will remove the adhesive baseplate wafer whenever they need to change the bag (this may be multiple times a day, although this number can be reduced, for example, to once a day, if the user can drain and clean the bag several times a day via the spout 1400). FIG. 4 shows an exploded view of the baseplate 1200 and the collection bag 1300 (including a draining spout 1400). FIGS. 5a to 5d show the baseplate itself in more detail. The baseplate 1200 is formed of at least three layers (as shown in FIG. 5c), which combine to form the wafer: a flange layer 1220, a central layer 1230, and a release liner 1240. The release liner 1240 is configured to be peeled off by a user to reveal the central layer 1230, which can then be brought in contact with the skin of a user surrounding a stoma, to attach the assembly 1000 to the peristomal skin.

As the central layer 1230 makes contact with the skin of the user over an extended period of time, it is formed of a skin-compatible material, which may, for example, be configured to regulate excess moisture and maintain healthy skin. The central layer 1230 may therefore be formed of a hydrocolloid, which can provide adhesion while offering skin-compatible properties. As the central layer 1230 is configured to adhere to the peristomal skin, it preferably provides skin compatible and soothing properties, such as regulating excess moisture and maintaining healthy skin. The central layer 1230 will therefore have thickness required to achieve this, typically between 0.7 mm and 1.5 mm, preferably between 0.8 mm and 1.2 mm, and more preferably approximately 1 mm. The flange layer 1220 complements the central layer 1230 by enhancing adhesion. The flange layer 1220 will therefore typically comprise a different adhesive than the central later 1230, which will typically provide stronger adhesion. The majority of the surface area of the baseplate 1200 in contact with the peristomal skin is the central layer 1230. However, the central layer 1230 has a diameter (or equivalent dimension) smaller than that of the flange layer 1220 such that the flange layer 1220 provides further adhesion to the skin around the central layer 1230.

Some of the surface area of the flange layer 1220 near its periphery therefore makes contact with the skin of a user around the stoma, forming a peripheral adhesive flange. The surface of the flange layer 1220 facing the user is configured to provide additional tack strength for improved adherence of the baseplate 1200 to the user. It must therefore be formed of a material with good adhesive properties, but which is also compatible with the skin. The adhesive may typically have a stronger adhesion strength than that of the central layer 1230. The flange layer 1220 may be formed from soft silicone adhesive, gentle acrylic adhesive, thin absorbent skin adhesive, non-woven silicone, polyolefin film, or a hydrocolloid (for example, on a polyurethane carrier). The flange layer 1220 has a smaller thickness that the central layer 1230, the flange layer 1220 having a typical thickness of between 250 and 500 microns, preferably between 250 and 400 microns, and more preferably between 275 and 375 microns. The positioning of the flange layer 1220 over and beyond the thicker central layer 1230 leads to an effectively ‘tapered’ edge to the baseplate, which can aid in fit, flexural properties, and help to resist peeling of the edges. The flange layer 1220 may also preferably be formed of a material having greater flexibility than the central layer 1230. The flange layer 1220 is provided adjacent to the bag 1300 and so the surface facing the bag 1300 (the surface away from the user) is configured to attach to the bag 1300 (this may be via a bonding mechanism such as, for example, welding). The flange layer 1220 in particular is attached to a spacer ring 1332, which is formed of a semi-rigid thermoplastic polyurethane (TPU) material, and which in turn is connected or connectable to the back sheet 1312 of the collection bag 1300. In the embodiment as illustrated, the flange layer 1220 is bonded to the spacer ring 1332, which in turn is bonded to the back sheet 1312. However, in alternative embodiments, a spacer ring and bag may comprise a coupling formation configured to allow reversible engagement and disconnection. The bonding method between the spacer ring 1332 and the flange layer 1220 of the embodiment as illustrated is chosen in dependence on the material of the flange layer; for example, welding may be used between a flange layer formed of a polyolefin film, while an adhesive may be used for other materials.

The anatomy of a user in the region of a stoma is unlikely to be a uniformly flat surface. Additionally, the user will move while wearing the ostomy bag assembly 1000. This can cause adhesive baseplates to wrinkle and become unstuck, which is insecure and can lead to leaks or loss of comfort for the user. The central layer 1230 has a non-circular outer edge shape. This is to improve conformity to the contours of a user's body. The central layer 1230 has this shape to help accommodate it having a greater thickness than the flange layer 1220. As illustrated in FIG. 5b, the central layer 1230 is formed as a flat disc having an overall circular shape (in that the outermost points of the layer form a circle). It has an approximate diameter in the range of 7 to 15 cm, preferably in the range of 8 to 12 cm, preferably approximately 10 cm. It comprises a central aperture 1210 with a diameter in the range of 1 to 5 cm, for example approximately 1.5 cm (this can be varied depending on the needs of the particular user). Around the outer edge or periphery, the layer comprises a plurality of formations (or ‘petals’) 1232, which protrude outwards from the centre. Each formation 1232 comprises an overhanging portion on the outer edge ('overhanging' in that it is a portion which extends/protrudes beyond the formation, where there is a gap between the extending portion and the main body of the central layer 1230). Between the overhanging portions on each of the formations, there are cut-outs 1234. The cut-outs 1234 separate the overhanging portions of the formations 1232 from the main body of the layer 1230. Providing separate formations 1232 which are able to move independently or at least partially independently of one another can assist in improving conformity of the baseplate 1200 to body/abdominal contours. It also improves the resistance to wrinkling which can be caused by the movements of a user.

The outer (peripheral) edge of each of the formations 1232 is curved, which can help to prevent lifting of the edges. In the illustrated embodiment, the radius of curvature of the outer edge of each of the formations 1232 is approximately 4 cm, which is approximately 80% of the radius of the overall central layer 1230. Additionally, the overhanging portions of each of the formations 1232 extend outwardly in a ‘swept’ arrangement, not simply in a radial direction, thereby forming a ‘pinwheel’ outer peripheral shape. In the embodiment as illustrated, the length of each formation 1232 from the centre of the layer 1230 (i.e. the centre of the aperture 1210) varies from approximately 5 cm to approximately 4.2 cm over a span of 72° (i.e. one fifth of the circumference of the circle). As such, the radius of the layer varies by approximately 10% to 20%, and preferably approximately 15% across each formation 1232. This means that central layer 1230 has rotational symmetry but not reflective symmetry. The largest width, at the overhanging portion, occurs in the close vicinity of the adjacent cut-out 1234. This can aid in increasing the ‘effective length’ of the cut-out 1234, which can assist in allowing the formations 1232 to move independently, without requiring the cut-outs 1234 to extend too far towards the centre of the layer 1230.

Each of the cut-outs 1234 extends into the layer 1230 a distance of approximately 1 cm, so approximately 20% of the total radius of the layer 1230 (although this may be within the range of 10 to 30%, and preferably 15 to 25%). The cut-outs span a segment of the layer 1230 of approximately 2° to 6°, preferably 3° to 5°, more preferably approximately 4°. If cut-outs 1234 were to extend close to the centre, causing the path between the aperture 1210 and the cut-outs 1234 to be small, there is an increased risk that leaks can occur. However, the cut-outs 1234 need to be of sufficient length that the formations 1232 can move independently of one another to the greatest extent possible, to conform over uneven body surfaces and withstand body movement. The extension of the overhanging portions of the formations 1232 and the cut-outs 1234 at an angle comprising both radial and tangential components means that the cut-outs 1234 have a greater effective length. This aids in separating the formations 1232, allowing them to move at least partially independently, without the need for the cut-outs 1234 to extend too close to the centre. Therefore, providing the cut-outs 1234 at an acute angle to the radial direction facilitates increased separation of the formations 1232 (which improves conformity to contours), while avoiding the risk of leaks occurring.

Each cut-out 1234 has a linear edge and a rounded end, with a radius of curvature of approximately between 2 and 3 mm, preferably between 2 and 2.5 mm, more preferably approximately 2.25 mm. These rounded ends can help to prevent tears occurring at the ends of the cut-outs 1234. They can also assist in maintaining adhesion by avoiding any sharp angles which could provide initiation points for wrinkles. The overhanging portion of each formation 1232 also has a rounded end (at the point where it extends to the greatest radius from the centre). The rounded end has a radius of curvature of approximately between 2 and 4 mm, preferably approximately 3 mm. The rounded edges can help to prevent lifting of the formations 1232 during use.

The flange layer 1220 is formed as a thin, generally flat, circular disc, which extends beyond the circumference of the central layer 1230, thus ensuring maximum adhering surface area. The flange layer 1220 of the illustrated embodiment is approximately 12 cm in diameter, but may preferably be in the range of 10 cm to 15 cm, preferably in the range of 11 cm to 13 cm. The flange layer 1220 conforms over the thicker central layer 1230 itself, so that the two layers combine to form a continuous linear surface on the side facing the user (the surface as shown in FIG. 5b). The surface of the flange layer 1220 opposite the user (adjacent the collection bag 1300) will have contours corresponding to the shape of the central layer 1230 underneath the flange layer 1220, including a bevel 1222 corresponding to the periphery of the central layer 1230 (as can be seen in FIG. 5d). The surface which makes contact with the skin should have an approximately linear surface to maximise the adhesion surface area and avoid air pockets between the baseplate and the skin as much as possible, as these can cause the baseplate 1200 to become loose or unstuck.

The release layer 1240 has a generally circular flat shape of diameter equal to, or slightly larger than, the flange layer 1220. It may comprise an aperture aligned with the aperture 1210 formed by the central layer 1230 and the flange layer 1220. The release liner 1240 may comprise a release tab 1242 which extends beyond its general circular diameter (and more importantly, the outer edge of the other layers), to provide the user with a gripping point with which to peel the release layer 1240 away from the other layers. The release layer 1240 may have a diameter slightly greater than that of the flange layer 1220 to further improve the ease with which it can be peeled off. It may have a diameter between 10 cm and 16 cm, preferably between 11 cm and 13 cm, more preferably approximately 2 cm. The release layer has a thickness of approximately 0.1 mm to 0.2 mm, and preferably approximately 0.15 mm. The release layer may also be printable; for example, this can allow for/provide an indication of different aperture diameters, so that a user can choose the appropriate size and cut the baseplate aperture to size as desired. Additionally, a user can cut the baseplate aperture to an irregular shape, in order to accommodate their particular requirements.

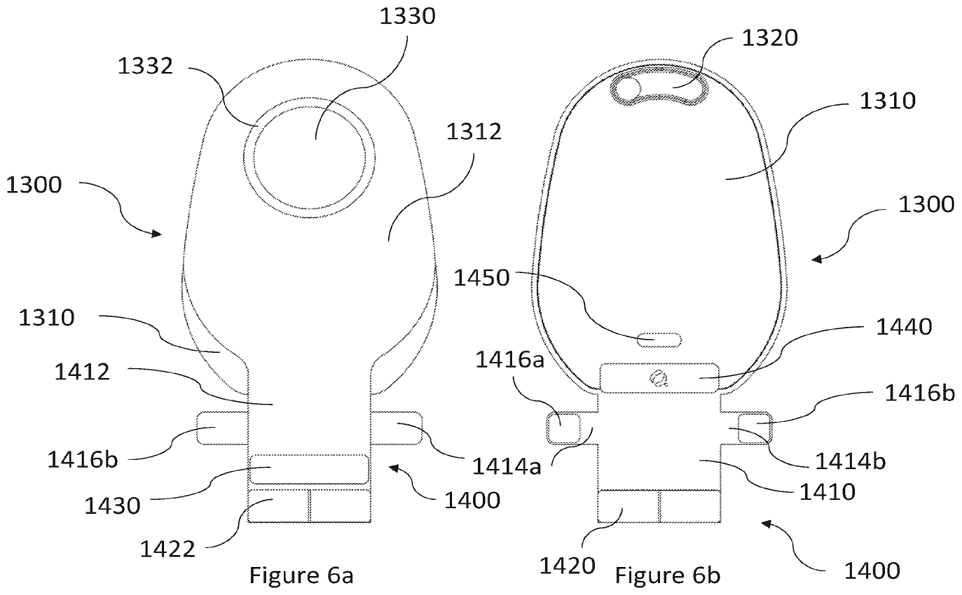

FIGS. 6a and 6b show a rear view and front view, respectively, of a collection bag 1300 of the present invention (where the front view refers to the side facing outwards when worn by a user). The bag 1300 is formed of a front sheet 1310 and a back sheet 1312 welded together to form a generally planar pouch. The front sheet 1310 and back sheet 1312 are formed of a waterproof material, so that bodily wastes cannot penetrate through the bag 1300. The material is preferably flexible and resilient. The material is also wipe-clean, to allow cleaning and/or disinfection. The bag 1300 may therefore be formed of a plastic material, such as flexible medical grade thermoplastic polyurethane (TPU). The material may also be soft-touch to enhance comfort.

The back sheet 1312 has a generally oval shape, the longer dimension configured to sit vertically on a user's body in use. The width of the back sheet 1312 is greater at one end. This wider end is configured to sit lower when in use, such that bodily wastes are encouraged to collect in this region. Extending from this wider, lower region, is an elongated portion 1412, which forms part of the spout 1400.

The front sheet 1310 is formed in a similar shape, but the wider end of the oval extends a greater distance along the length of the pouch. This forms a tapered pouch in which waste can collect. The front sheet 1310 and back sheet 1312 are welded together around the perimeter of the back sheet 1312. The larger surface area of the front sheet 1310 acts to smooth the surface of the bag assembly, so that even when it is half full or full, no bulge is obvious under a wearer's clothes. An elongated portion 1410 extends from the lower region (in the orientation when worn, in use), corresponding to the elongated portion 1412 of the back sheet. The front sheet elongated portion 1410 and the back sheet elongated portion 1412 together form the spout 1400. The two sheets are not bonded together at the ends of the elongated portions 1410 and 1412 (i.e. the end distal from the main body the bag 1300), forming an open-ended spout 1400.

The back sheet 1312 comprises an aperture 1330. The spacer ring 1332 is provided around the aperture and bonded to both the back sheet 1312 and the baseplate 1200. The front sheet 1310 and back sheet 1312 are welded together such that there is a continuous fluid conduit from the aperture to a pouch formed between the front and back sheets, through and out of the spout 1400. The bag aperture 1330 is in fluid connection with the baseplate aperture 1210, so that the bag 1300, including the spout 1400, can be fluidly connected to a stoma.

The front sheet 1310 further comprises a filter vent 1320. This is located near the end of the pouch which is located at the top of the ostomy device when worn by a user. The filter vent 1320 is configured to allow venting of gases while filtering odours. Sufficient venting of gas from the ostomy bag 1300 is important to prevent gas from collecting in the bag, causing it to ‘balloon’, which can be uncomfortable for the wearer and can cause the bag to become visible under the wearer's clothing. In a worst-case scenario, the bag 1300 could even pull away and off from the user's body. The vent 1320 also provides a filtering capability, configured to deodorise any gases removed from the bag 1300 before releasing them into the air.

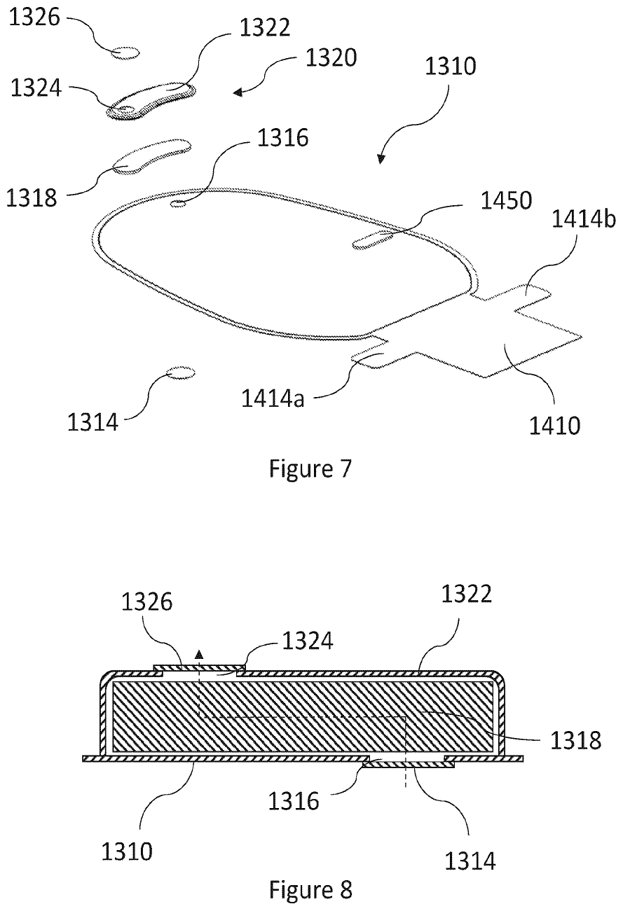

FIG. 7 shows a perspective exploded view of the component parts of the front sheet 1310. A vent aperture 1316 is provided in the sheet 1310, across which is provided a first semi-permeable membrane 1314. Over the first semi-permeable membrane 1314 is provided a filter 1318. The filter 1318 is formed as a strip having a longer length than width or depth, and one end of the strip (along the length) covers the first semi-permeable membrane 1314. Provided over the filter 1318 is a filter cover 1322. The filter cover 1322 comprises a further vent aperture 1324, across which is provided a further semi-permeable membrane 1326. The further vent aperture 1324 and further semi-permeable membrane 1326 are located at the opposite end along the length of the strip to the first vent aperture 1316 and first semi-permeable membrane 1314. The filter cover 1322 has a shape corresponding to the filter 1318 so that it covers it completely. The filter cover 1322 is formed of a rigid gas-impermeable material (such as TPU), and is welded around the perimeter of the filter 1318. Accordingly, the filter 1318 is sealed within the cover 1322, and the only path of travel for gases is through the first semi-permeable membrane 1314, along the length of the filter 1318, and through the second semi-permeable membrane 1326. FIG. 8 shows a schematic cross-sectional view of the filter vent 1320, the dashed arrow indicating the path of travel of a gas. This arrangement therefore provides an in-plane filtering gas flow path.

The first semi-permeable membrane 1314 provides an internal semi-permeable membrane to prevent any unwanted ingress of the bodily wastes contained within the bag into the filter 1318. This is to prevent the filter 1318 from becoming clogged. The first semi-permeable membrane 1314 is made from a low friction material to prevent waste material from becoming attached to the surface, so that gas can continue to pass through the first semi-permeable membrane 1314 towards the filter 1318. Preferably, the membrane comprises a hydrophobic and/or oleophobic coating (at least) on the surface facing within the bag. The first semi-permeable membrane 1314 is welded to the front sheet 1310 around its perimeter in order to form a secure and gas-tight bond.

Once the gas has passed through the first semi-permeable membrane 1314, it enters a chamber formed by the filter cover 1322, within which is provided the filter 1318. The filter 1318 is arranged so that the length is positioned within the plane of the bag 1300 (so approximately parallel to the surface of the wearer's body when in use). The first semi-permeable membrane 1314 is located at one end of the filter 1318 along the elongate direction, while the second semi-permeable membrane 1326 is located at the opposite end along the elongate direction. This means that the gas passes through the filter along the longer dimension, providing ‘in-plane’ filtering. The optimised flow path along the length of the filter 1318 ensures maximum exposure to a deodorising material, while ensuring the filtering arrangement is not bulky on a user's body. In the illustrated embodiment, the filter is formed as an arc, which increases the length of the flow path in a space-efficient manner, However, it should be understood that other shapes providing a generally elongate strip could also be used.

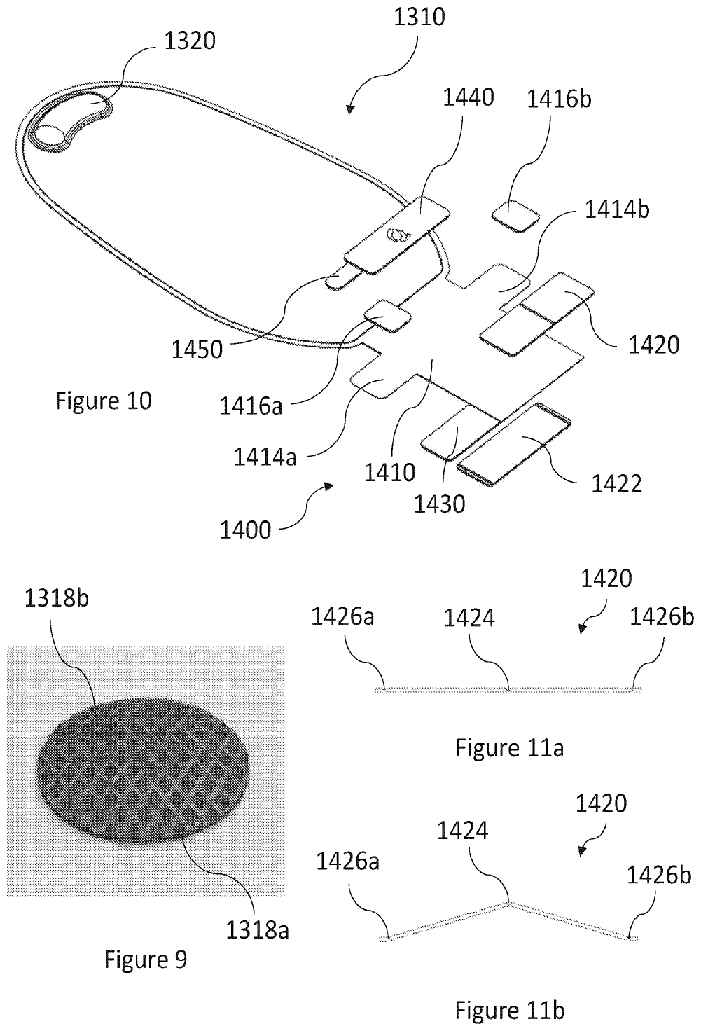

In this exemplary implementation, a deodorising carbon material is used, such as material comprising activated carbon. The filter 1318 may be formed as a compound weave of activated carbon cloth in order to encourage the ‘in-plane’ deodorisation. The filter 1318 is bonded to the surface of the front sheet 1310 and to the filter cover 1322. The bonding mechanism must be secure but must also allow gas to pass through relatively uninhibitedly. In order to achieve this, glue is applied in a diamond lattice pattern. This is illustrated in FIG. 9, which shows activated carbon cloth 1318a with a glue lattice 1318b applied to the surface. The glue used may, for example, be ethylene vinyl acetate (EVA) glue.

After the gas has passed through the filter 1318, it passes through the second semi-permeable membrane 1326, and is expelled from the device. As the second semi-permeable membrane 1326 is provided on the external surface of the ostomy bag, it is configured to prevent unwanted ingress of external matter into the filter, for example water when a user showers. In order to achieve this, the second semi-permeable membrane 1326 also has a hydrophobic and/or oleophobic coating (at least) on the surface facing externally when in use. The second semi-permeable membrane 1326 is welded to the filter cover 1322 around its perimeter in order to form a secure and gas-tight bond.

The filter 1318 should be of a sufficient length to facilitate maximum exposure of any gases to the deodorising material, but it must not be of such a length that gas flow is hindered, as this can make the bag more susceptible to ‘ballooning’. The length required to achieve this will be dependent on the particular properties of the filter material chosen. Typically, the length of the filter will be between 2 cm and 10 cm, preferably between 3 cm and 8 cm.

FIG. 10 shows a further perspective view of the front sheet 1310, with an exploded view of the component parts of the spout system 1400. The spout system 1400 is configured to be such that a user can unroll it to an extended configuration (as shown in FIGS. 1a and 1b), in which the distal end of the spout is open such that a user can drain waste matter from the bag 1300. The spout 1400 is configured such that a user can then securely roll it back up again to a rolled-up configuration (as shown in FIGS. 2a and 2b), where the bag 1300 is sealed and can collect bodily wastes securely.

At the open end of the spout 1400 (i.e. the distal end) is provided a first tailpiece support 1420 and a second tailpiece support 1422. The first tailpiece support 1420 is provided on the elongated portion 1410 of the front sheet 1310, and the second tailpiece support 1422 is provided on the elongated portion 1412 of the back sheet 1312. FIG. 11a shows a side view of the first tailpiece 1420 support in a flat configuration while FIG. 11b shows a side view of the first tailpiece 1420 support in a bent configuration. The first tailpiece is formed as a strip of rigid plastic material and comprises three hinges: a central hinge 1424 bends in a first direction, and two hinges at either end 1426a, 1426b which bend in the opposite direction. The hinges 1424, 1426a and 1426b are formed as grooves in the rigid material, which leaves a line in which the thickness of the tailpiece is reduced, along which it can fold (providing a fold-line). In the illustrated embodiment, these are U-grooves. As shown in FIG. 11b, this hinge arrangement allows two central parts of the tailpiece 1420 to form an angle while the ends remain at the same orientation. The second tailpiece support 1422 has the same construction and is provided on the elongated portion 1412 of the back sheet 1312 in a symmetrical orientation. This allows the first tailpiece support 1420 to bend in one direction and the second tailpiece support 1422 to bend in the other direction, such that they form an open end. The two tailpiece supports 1420, 1422 thus provide support and structure to the opening of the spout 1400. When the spout 1400 is opened to allow waste matter to be drained, the two tailpiece supports 1420, 1422 provide structural rigidity which can hold the end open, improving the ease with which the bag can be cleaned. When the user wishes to close the spout 1400, roll it up and secure it, the two tailpiece supports 1420, 1422 remain flat, and the structural rigidity of the ends in this configuration can assist a user in rolling the spout.

The spout 1400 comprises two wings, 1414a and 1414b, extending from either side of the elongated portion 1410 of the front sheet 1310. On each of the wings is located adhesive tape to provide wing fasteners 1416a and 1416b (which for example are formed of loop tape of a hook and loop fastener, such as Velcro™). The wing fasteners 1416a and 1416b are provided on the front sheet 1310. A tail fastener 1430 is provided on the elongated portion 1412 of the back sheet 1312, at a position along the length intermediate between the tailpiece supports 1420, 1422 and the wings 1414a and 1414b. The tail fastener 1430 is formed of adhesive tape such as hook tape (confirgured to engage with the loop tape of the wing fasteners 1416a and 1416b). The tail fastener 1430 is configured to engage with and adhere to the wing fasteners 1416a and 1416b when the spout is rolled up and the wings 1414a and 1414b are folded in.

At the boundary between the main body of the bag and the elongated portion 1410 on the front sheet 1310 is provided a pressure strip 1440. The pressure strip 1440 is formed as a rigid or semi-rigid strip which spans across the width of the spout 1400. This is typically formed from a plastic material such as thermoplastic polyurethane (TPU). The pressure strip 1440 allows a user to apply pressure in an even distribution along the bottom opening of the bag 1300, between the main body of the bag and the spout 1400. This prevents any matter from passing between the main body and the spout 1400, thereby allowing users to control the flow of output out of the spout 1400. This can also improve the ease with which the spout 1400 can be cleaned, for example by allowing users to prevent further ingress into the spout 1400, thereby allowing the spout 1400 to be wiped clean and disinfected before it is rolled up again. The pressure strip may also provide additional security that matter will not leak out when the spout 1400 is in the rolled-up configuration.

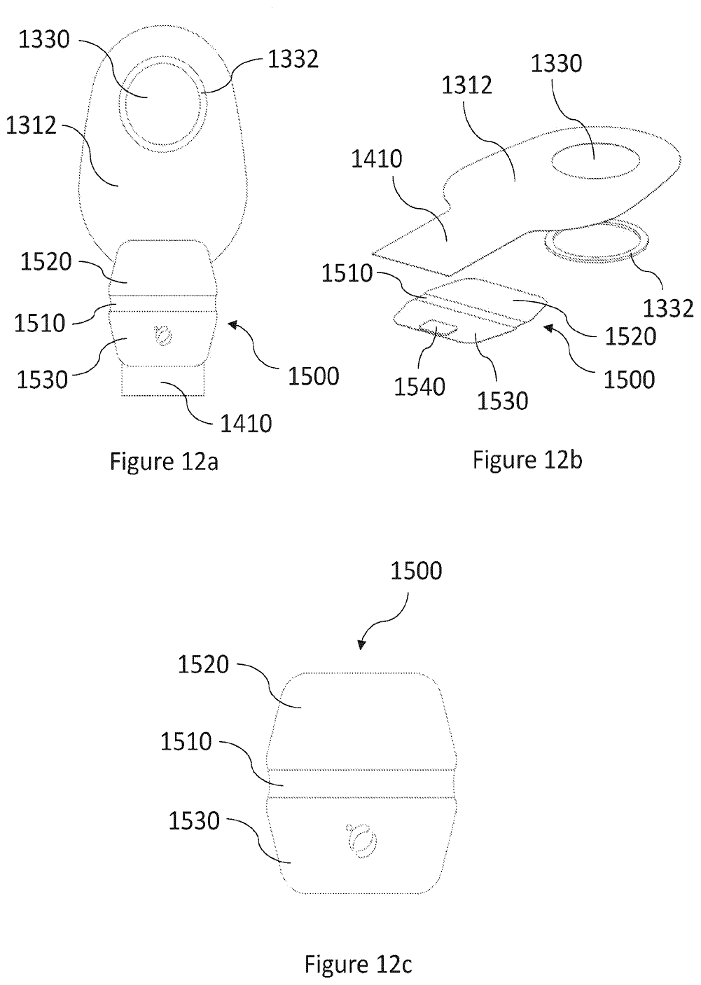

A retaining flap 1500 is also provided, which is configured to wrap around and secure the spout 1400 when in a rolled-up configuration. The retaining flap 1500 can also smooth over the rolled-up spout 1400, so that it is less obvious under a user's clothes. As shown in FIG. 12c, the retaining flap 1500 comprises a back flap portion 1520, a front flap portion 1530 and a joining section 1510. The three portions may be formed of a single piece of material, or the joining section 1510 may be bonded to the back flap portion 1520 and the front flap portion 1530 to connect them. The back flap portion 1520 is bonded to the main body of the back sheet 1312, just above the join with the elongated portion 1412, which forms part of the spout 1400, so that the spout 1400 can freely roll up. As can be seen in FIGS. 1a, 1b and 12a, the spout 1400 extends beyond the retaining flap 1500 when in the extended (unrolled) configuration.

The front flap portion 1530 comprises a first flap fastener 1540, which is configured to engage with and connect to a second flap fastener 1450, which is located on the main body of the front sheet 1310. The first flap fastener 1540 and the second flap fastener 1450 may typically be provided as a hook and loop fastener system.

In order to apply the ostomy bag system of the present invention, the user will remove the release liner 1240 from the baseplate 1200 in order to reveal the adhesive surfaces of the central layer 1230 and the flange layer 1220. The user can then place the adhesive surfaces on the peristomal skin surface, aligning the aperture 1210 with the stoma.

The bag system may be provided with the spout 1400 in the rolled-up configuration, or the user may need to secure it in this configuration. This can be achieved by the user taking hold of the tailpiece supports 1420 and 1422, which have greater rigidity than the material of the front sheet 1310 and back sheet 1312. The user ensures that the tailpiece supports 1420 and 1422 are in a flat configuration (as shown in FIG. 11a), and then, holding them together, folds the tailpiece supports 1420 and 1422 upwards onto the surface of the elongated portions 1410 of the front sheet 1310. The user continues to fold until the tail fastener 1430 (which is located on the elongate portion 1412 of the back sheet 1312) is then brought onto the top surface, facing outwardly from the user when in use. The user then folds the wings 1414a, 1414b towards the spout, causing the wing fasteners 1416a and 1416b to make contact with and engage the tail fastener 1430. This secures the spout 1400 in a ‘rolled-up’ configuration. The user can then fold the front portion 1530 of the retaining flap 500 around and over the rolled-up spout, the joining section 1510 acting generally as the hinge point. The user brings the first flap fastener 1540 on the front flap portion 1530 up towards the second flap fastener 1450 on the body of the front sheet 1310 of the bag. The first flap fastener 1540 and the second flap fastener 1450 connect and engage to secure the retaining flap 1500. The user can then leave the bag in this secured configuration, allowing it to collect bodily waste.

If the user wishes to drain the bag, they can reverse the process as described above to un-roll the spout 1400. The user can release the retaining flap 1500 by releasing the first flap fastener 1540 and the second flap fastener 1450. The user can then release the wing fasteners 1416a and 1416b from engagement with the tail fastener 1430. They can then unroll the spout 1400, and open the distal end by pressing on the ends of the tailpiece supports 1420 and 1422 to move them from the flat configuration (as shown in FIG. 11a) to the bent configuration (as shown in FIG. 11b). This provides a rigid ring opening at the distal end of the spout, through which waste matter from the bag can be drained. Once the user has drained the waste matter from the bag as desired, they can close the opening between the body of the bag and the spout 1400 by applying pressure to the pressure strip 1440. This prevents any further waste matter from entering the spout 1400, enabling the user to clean the spout 1400. The user can then repeat the rolling procedure as described above.

### Alternatives

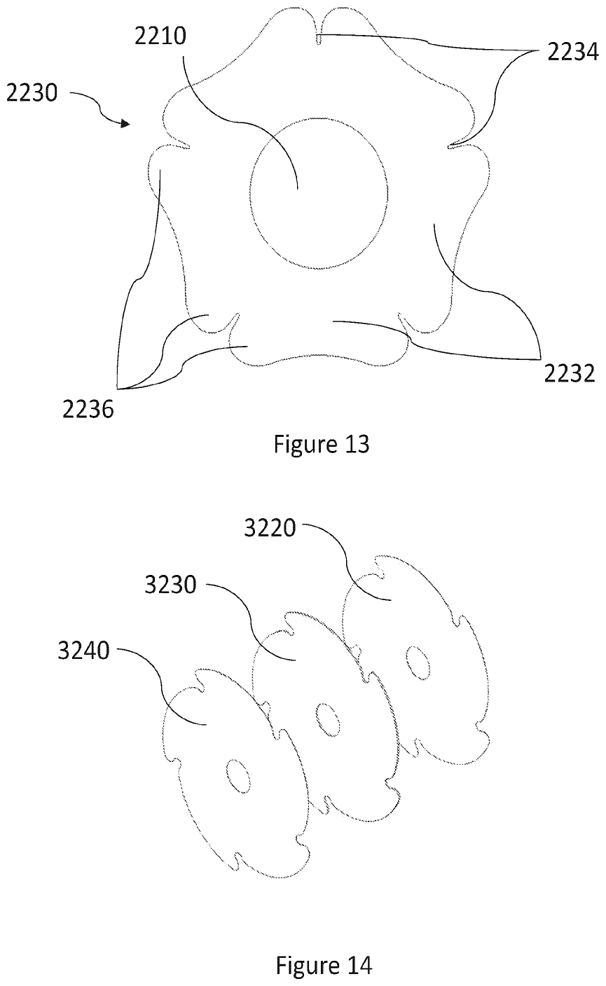

FIG. 13 shows an alternative shape for the central layer of the baseplate. The further embodiment of the central layer 2230 also comprises an aperture 2210, which is configured to accommodate a stoma, and formations (petals) 2232 which extend outwardly from the centre. The formations 2232 are separated by cut-outs 2234. Each formation is shaped to have a concave outer surface, such that both edges of the formation (petal) extend to a greater radius than the central portion of each formation (petal). This has the effect of increasing the effective length of the cut-outs 2234 without requiring that they extend too close to the aperture 2210 (which could lead to an increased likelihood of leaks between the aperture and the cut-outs). This improves the separation of the formations 2232, so that they can move partially independently from one another. The ability of the formations 2232 to move partially independently from one another can improve the conformability of the layer over an uneven surface, and prevent the layer from becoming unstuck when the user moves and twists during everyday movement.

The central layer of the baseplate may also be formed of further alternative shapes which comprise cut-outs between formations. In order to achieve good separation of the formations, such that they can move partially independently of one another, the formations are preferably configured such that the length of the cut-outs is maximised while allowing for sufficient material between the end of the cut-out and the aperture (in order to prevent leaks from occurring along a pathway). In order to achieve this, one or both sides of the peripheral edge of the formations extends to a larger distance away from the centre of the baseplate than the centre of the peripheral edge of the formation. For example, the configuration as shown in FIG. 5 achieves this via a ‘swept’ arrangement (i.e. the overhanging portions of the formations and the cut-outs extend in a direction with a tangential component, not directly aligned with the radial direction towards the centre of the layer). The configuration as shown in FIG. 13 achieves this via the peripheral edges of the formations having a concave shape, such that the sides extend to a greater radius than the centre (thereby forming the ‘overhanging portion’ of this embodiment).

The baseplate has been described as approximating a circular shape; however, it should be understood that the required properties could also be achieved via implementation as alternative shapes.

The layers of the baseplate wafer may alternatively have different configurations. For example, as shown in FIG. 14, the layers may all have the same formation/petal configuration. The central layer may also have a thicker central region. In some embodiments, a greater number of layers may also be provided. The wafer in general, and the flange layer or layers in particular may comprise more than material, for example acrylic tapes may be provided around the periphery to enhance adhesion.

The baseplate wafer as described and illustrated is formed as having a generally planar, flat configuration. However, the baseplate may alternatively be provided as having a concave or convex configuration.

The illustrated embodiment comprises a spacer ring bonded to the baseplate wafer to facilitate connection to the ostomy bag. It should, however, be understood that this is exemplary only and alternative connections may be implemented between the baseplate wafer and the ostomy bag. For example, the baseplate wafer may be directly connectable or connected (for example, bonded) to the ostomy bag. Alternatively, a reversible connection mechanism may be provided between the baseplate wafer and the ostomy bag.

It should also be understood that the retaining flap 1500 is optional, and the bag may be provided without it. The retaining flap 1500 may also be provided as a separate element which is reversibly attachable and detachable to the ostomy bag.

Although the illustrated embodiment shows the glue provided a diamond lattice pattern, it should be understood that the glue may be provided in alternative discontinuous patterns. The discontinuous nature of the pattern allows gas to pass through, but may be implemented in different patterns or arrangements comprising spaces or pores in the glue layer.

It will be understood that the present invention has been described above purely by way of example, and modifications of detail can be made within the scope of the invention.

Each feature disclosed in the description, and (where appropriate) the claims and drawings may be provided independently or in any appropriate combination. For example, the adhesive baseplate and/or the filter may be provided on a different ostomy bag arrangement. In some implementations, an ostomy bag arrangement may be provided comprising the baseplate and filter assemblies as described above, without the cleaning spout. For such an embodiment, the expected behaviour is that users will remove the adhesive and the whole assembly whenever the bag is full and needs changing.

It should be understood that the pressure strip arrangement is optional, and the adhesive baseplate and/or the filter may be provided without the pressure strip.

Reference numerals appearing in the claims are by way of illustration only and shall have no limiting effect on the scope of the claims.

1. An adhesive wafer for an ostomy bag system, comprising a layer, the layer comprising a plurality of formations at an outer part of the layer, wherein each formation comprises an overhanging portion.

2. The adhesive wafer of claim 1, wherein the layer comprises a plurality of cut-outs extending into the layer, wherein at least a portion of each of the plurality of cut-outs extends into the layer in a direction which is not directly towards a centre of the layer.

3. The adhesive wafer of claim 2, wherein each overhanging portion is separated from the layer by a respective cut-out.

4. An adhesive wafer for an ostomy bag system, comprising a layer, the layer comprising a plurality of cut-outs extending into the layer, wherein at least a portion of each of the plurality of cut-outs extends into the layer in a direction which is not directly towards a centre of the layer.

5. An adhesive wafer according to claim 4, wherein the layer comprises a plurality of formations at an outer part of the layer, wherein each formation comprises an overhanging portion.

6. The adhesive wafer of any of claim 1 to 3 or 5, wherein an outer edge of each of the formations is shaped as part of a spiral.

7. The adhesive wafer of any of claim 1 to 3 or 5 or 6, wherein the formations are arranged evenly around the layer, preferably wherein the formations are arranged so as to be rotationally symmetrical.

8. The adhesive wafer of any of claims 1 to 3 or 5 to 7, wherein the overhanging portions have a rounded end, preferably wherein the overhanging portions have a partially circular end, more preferably wherein the partially circular end has a radius of curvature of between 2 mm and 4 mm, yet more preferably of approximately 3 mm.

9. The adhesive wafer of any of claims 3 to 5 or any of claims 6 to 8 when dependent on any of claims 3 to 5, wherein the cut-outs have curved ends, preferably wherein the curved ends have a generally semi-circular shape, more preferably wherein the curved ends have a radius of curvature of between 2 and 3 mm, preferably between 2 and 2.5 mm.

10. The adhesive wafer of any preceding claim, wherein the outermost points of the layer are arranged along a circle.

11. The adhesive wafer of any preceding claim, wherein the cut-outs extend into the layer in a direction which is at an acute angle to a radius of the layer, preferably wherein said angle is between 15 and 45 degrees, more preferably wherein said angle is between 25 and 35 degrees, yet more preferably wherein said angle is approximately 30 degrees.

12. The adhesive wafer of claim 11, wherein the cut-outs extend into the layer by not more than 30% of a width of the layer, preferably between 15 to 25%, more preferably by approximately 20%.

13. The adhesive wafer of any preceding claim, further comprising an aperture located at a central position on the layer.

14. An adhesive wafer of any preceding claim, further comprising a further layer, wherein the layer has a greater thickness than the further layer, and/or the further layer has a dimension greater than that of the layer, such that it extends beyond the outer part of the layer.

15. An adhesive wafer for an ostomy bag system, formed as a wafer comprising at least a first layer and a second layer, wherein the first layer comprises a plurality of formations at an outer part of the layer and the second layer has a dimension greater than that of the first layer, such that it extends beyond the outer part of the first layer, preferably wherein the first layer has a greater thickness than the second layer.

16. The adhesive wafer of any preceding claim, wherein the further layer or second layer comprises an adhesive, preferably wherein the layer or first layer comprises a different adhesive, more preferably wherein the layer or first layer comprises a hydrocolloid.

17. A filter system for an ostomy bag comprising: a first aperture provided on a first surface, a second aperture provided on a second surface; and a filter, wherein the filter is provided between the first surface and the second surface and between the first aperture and the second aperture, such that the first aperture and the second aperture are located at different ends of a length of the filter.

18. The filter system of claim 17, wherein the first aperture and/or second aperture comprise a semi-permeable membrane, preferably having a low-friction coating.

19. The filter system of claim 17 or 18, wherein the filter comprises activated carbon.

20. The filter system of any of claims 17 to 19, wherein the filter is bonded to the first and/or second surface by a glue arranged in a lattice formation.

21. An ostomy bag, comprising means arranged to be held by a user so as to temporarily occlude an opening in the ostomy bag.

22. The ostomy bag of claim 21, wherein the means is a rigid or semi-rigid strip of material, preferably being mounted on an outer part of the ostomy bag and/or extending coincident to the width of the opening, more preferably such that when held by the user the formation applies pressure over the width of the opening.

23. The ostomy bag of claim 21 or 22, comprising a draining spout extending from the opening, wherein the draining spout is configured to be folded into a sealed configuration; preferably wherein the spout comprises wings which form part of a fastening mechanism.

24. The ostomy bag of claim 23, wherein a distal end of the spout is configured to be moveable between a flat configuration and an open configuration, preferably wherein the distal end of the spout is formed of a rigid or semi-rigid material; more preferably wherein the distal end comprises fold lines.

25. An ostomy bag system comprising at least one of:
the adhesive wafer of any of claims 1 to 16;
the filter system of any of claims 17 to 20; and
the ostomy bag of any of claims 21 to 24.
